SEQUENCE LISTING (14 sequences)

SEQ ID NO: 1 - DNA, 30 bp, Artificial Sequence
  Primer
ctgcaggaattcgaacgctgacgtcatcaa

SEQ ID NO: 2 - DNA, 62 bp, Artificial Sequence
  Primer
aaatctcttgaatttaacaatacccagctccggggatctgtggtctcatacagaacttataa

SEQ ID NO: 3 - DNA, 59 bp, Artificial Sequence
  Primer
ccatcgataagctttttttccaaaaaaggagctgggtattgttaaatctcttgaattta

SEQ ID NO: 4 - DNA, 21 bp, Artificial Sequence
  Primer
tgttaccagggaggagcagtg

SEQ ID NO: 5 - DNA, 21 bp, Artificial Sequence
  Primer
tcttgcccttcctttctgtca

SEQ ID NO: 6 - DNA, 27 bp, Artificial Sequence
  Primer
caggactctaatccagagtttaccttc

SEQ ID NO: 7 - DNA, 16 bp, Artificial Sequence
  Primer
gggatggctgccagca

SEQ ID NO: 8 - DNA, 22 bp, Artificial Sequence
  Primer
agcagccctacgaccaggccca

SEQ ID NO: 9 - DNA, 21 bp, Artificial Sequence
  Primer
gccaaactacagcgaactgga

SEQ ID NO: 10 - DNA, 21 bp, Artificial Sequence
  Primer
tgtggtatgacaggcatggag

SEQ ID NO: 11 - DNA, 31 bp, Artificial Sequence
  Primer
cacatacagtgattatttccccgtatctcta

SEQ ID NO: 12 - DNA, 40 bp, Homo sapiens
  misc_feature: human dEF1 mRNA
aaaaaatacaaaatacaaaacacacacacacacacacaca

SEQ ID NO: 13 - DNA, 40 bp, Homo sapiens
  misc_feature: Human SIP1 mRNA
gacggagttggagctgggtattgttaaaaactgcattatg

SEQ ID NO: 14 - DNA, 19 bp, Homo sapiens
  misc_feature: siRNA SIP1
ggagctgggtattgttaaa

(19) United States
(12) Patent Application Publication
(10) Pub. No.: US 2011/0104183 A1
(43) Pub. Date: May 5, 2011

(54) USE OF SIP1 AS DETERMINANT OF BREAST CANCER STEMNESS

(75) Inventors: Geert Berx, Linter-Wommersom (BE); Cindy Vandewalle, Gent (BE); Eric Raspe, Mouscron (BE)

(73) Assignees: VIB VZW, Gent (BE); UNIVERSITEIT GENT, Gent (BE)

(21) Appl. No.: 12/735,984

(22) PCT Filed: Feb. 26, 2009
(86) PCT No.: PCT/EP2009/052304

§ 371 Date: Nov. 16, 2010

Related U.S. Application Data
(60) Provisional application No. 61/067,511, filed on Feb. 27, 2008.

(51) Int. Cl. A61K 39/395; C12Q 1/68; C12N 5/095; A61K 31/7105; G01N 33/574; A61P 35/00

(52) U.S. Cl. 424/174.1; 435/6; 435/375; 514/44 A; 435/7.23

(57) ABSTRACT

The present invention relates to the diagnosis and treatment of cancer. More specifically, it relates to the use of SIP1 nucleic acid and/or protein for the detection of breast cancer stem cells, and the repression of the gene and/or the inactivation of the protein to repress the differentiation of cells into cancer cells and to inhibit metastasis of breast cancer tumors.

### CROSS-REFERENCE TO RELATED APPLICATION

This application is a National Stage filing under 35 U.S.C. §371 of International Patent Application Number PCT/EP2009/052304, filed Feb. 26, 2009, designating the United States of America, published on Sep. 3, 2009, in English as WO 2009/106578 A1, and claims priority to U.S. Ser. No. 61/067,511, filed Feb. 27, 2008.

### TECHNICAL FIELD

The present invention relates to the diagnosis and treatment of cancer. More specifically, it relates to the use of SIP1 nucleic acid and/or protein for the detection of breast cancer stem cells, and the repression of the gene and/or the inactivation of the protein to repress the differentiation of cells into cancer cells and to inhibit metastasis of breast cancer tumors.

### BACKGROUND

It is rare for a cancer patient to die due to the local effects of their primary tumor. Rather, it is the metastatic spread of tumor cells that is ultimately responsible for the vast majority of cancer morbidity and deaths. Understanding the cell and molecular biology of invasion and metastasis and the genetic changes that drive these processes represents one of the last great frontiers of exploratory cancer research. Therapies directed against metastatic cells hold the promise of clearing the body of tumor cells and curing the patient.

Despite the central importance of tumor metastasis for the clinical management of cancer, we are still a long way from understanding how tumor dissemination is orchestrated, be that at the molecular, cellular or organismic level. The mechanisms leading to the metastatic dissemination of tumor cells appear to be similar for many different types of cancer and are associated with multiple cellular processes. These include the transition of tumor cells from an epithelial, adhesive phenotype to cells with mesenchymal morphology and migratory and invasive capabilities, invasion into surrounding tissue, intravasation into blood or lymphatic vessels, survival and dissemination through the blood or lymphatic circulation, colonization of distant organs by adhesion to the vessel wall, extravasation and invasion into distant organ parenchyma, and finally metastatic outgrowth in the distant organ (Sleeman, 2000). Thus, metastasis is a highly complex problem with many facets. Several hypotheses have been developed over recent years to explain how this process, or aspects of it, is regulated. However, none of these explanations fully reconcile the available experimental data.

Recent ideas about the cellular basis of tumor growth (cancer stem cells) and the establishment by remote tumors of special permissive microenvironments in target organs prior to metastasis (metastatic niches) have the potential to radically change our view of the metastatic process. It has been established for many types of cancer that the bulk of cells that make up a tumor are derived from a small subpopulation of cancer stem cells (CSCs). CSCs are distinguished from the bulk population of tumor cells by their ability to successfully seed new tumors when implanted in low numbers into experimental animals. In contrast, the non-CSC population cannot initiate tumor growth in vivo even when implanted in high numbers (Dalerba et al., 2007). The concept of CSCs should have major consequences for our understanding of the dissemination and metastasis of solid tumors, as CSCs probably represent the only subpopulation of cells able to seed metastases successfully. However, this research area remains little explored and has not been integrated into current concepts that attempt to explain the process of metastasis. Furthermore, an emerging paradigm is that tumors are able to produce factors that induce the formation of so-called pre-metastatic niches in organs where metastases will ultimately develop (Kaplan et al., 2005; Hiratsuka et al., 2006). These pre-metastatic niches are thought to provide an appropriate and requisite environment for the development of secondary tumors. Again, the molecular and cellular mechanisms underlying the establishment of pre-metastatic niches remain poorly investigated.

Taken together, these observations indicate that our concept of the process of metastasis is still incomplete and needs a major overhaul. In particular, novel experimental approaches are needed that integrate newly emerging principles and ideas with the different hypotheses that have thus far been developed to explain the process of metastasis. This will facilitate the development of an improved and more accurate concept about the process of metastasis. In turn, this will have fundamental ramifications for the way in which novel anti-cancer therapies are designed, and most importantly should provide important new insights into how cancer and in particular metastatic disease can be successfully treated.

Smad-interacting protein (SIP1, also known as ZEB2, accession number NP_{—}055610; nucleic acid accession number NM_{—}014795) belongs to the δEF-1 of ZEB protein family. These proteins are characterized by a homeodomain flanked by two separated, highly conserved zinc finger clusters. Each zinc finger cluster can bind independently to sequences present in promoter regions of genes involved in differentiation and development, such as the E-cadherin promoter (Vandewalle et al., 2005). Binding of SIP1 to the E-cadherin promoter down-regulates E-cadherin expression (Comijn et al., 2001). In epithelial MDCK cells, this suppression of E-cadherin expression is accompanied by loss of aggregation and acquisition of invasive properties. Recently, Bindels et al. (2006) demonstrated that SIP1 regulates vimentin expression in epithelial cells, and suggested a role of SIP1 in cell migration and invasion of epithelial breast cancer.

The estrogen receptor-negative human breast cancer cell line MDAMB231 was isolated from a pleural effusion of a breast cancer patient suffering from widespread metastasis, years after removal of her primary tumor (Calleau et al., 1978). MDAMB231 is a heterogeneous cell population containing cell types with a morphology ranging from more epithelial-like to mesenchymal-like. This phenotypic variation is reflected at the molecular level as subclones, either obtained in vitro or in vivo through selection of subpopulations with different metastatic capabilities (Kang et al., 2003), showing significantly different gene expression profiles.

This cell line has been widely used as a model for invasion and metastasic breast cancer (Lacroix and Leclercq, 2004). The cell line shows a general mesenchymal-like phenotype, but is in fact highly heterogeneous and thus includes cells with various morphological characteristics. MDAMB231 cells are highly invasive in vitro and injection in nude mice leads to tumorigenicity and, depending on the site of injection, to aggressive behavior (Lacroix and Leclercq, 2004). The cells are E-cadherin negative and exhibit a high level of the mesenchymal marker vimentin. In order to examine the functional contribution of ZEB2/SIP1 to the dedifferentiated state and invasive and metastatic potential of this breast cancer cell line, we established an RNAi-based stable ZEB2/SIP1 knock-down in MDAMB231 and analyzed the functional and molecular consequences in vitro as well as in vivo.

Surprisingly, contrary to what could be expected, the expression of SIP1 is limited to a minority of cancer cells and is a useful marker for cancer stem cells. Indeed, SIP1 is expressed in the normal mammary gland in distinct cells surrounding the normal ducts, but SIP1 is only expressed in a minority of breast cancer cells in a xenografted human tumor model as well as in human breast cancers. Even more surprisingly, notwithstanding the limited number in which SIP1 is expressed, SIP1 repression in human aggressive breast cancer cells dramatically attenuates tumor growth. As a consequence of the differentiation of cancer stem cells, which lose as such their sternness, SIP1 repression induces the expression of epithelial differentiation and aggregation and down-regulates the migratory and invasive capacity, consequently blocking migrating cancer stem cells.

### DISCLOSURE OF THE INVENTION

A first aspect of the invention is the use of the SIP1 gene, and/or the SIP1-encoded protein, as a marker for breast cancer stem cells. More specifically, it is the use of the SIP1 gene and/or the SIP1-encoded protein for the detection of breast cancer stem cells. Indeed, within a population of cancer cells, cells expressing SIP1 behave as cancer stem cells. Methods to detect the expression of a gene are known to the person skilled in the art and include, but are not limited to, hybridization and PCR. The protein can be detected by, as a non-limiting example, the use of anti-SIP1 protein antibodies.

Another aspect of the invention is the use of the SIP1 gene repression, and or the inactivation of SIP1 protein to inactivate breast cancer stem cells. “Inactivation of breast cancer stem cells,” as used herein, means that the SIP1-expressing cells lose their stemness, resulting in a limitation of the tumor development and a repression of metastasis.

Methods for gene repression are known to the person skilled in the art and include, but are not limited to, SIP1-specific antisense RNA or SIP1-specific RNAi. Methods to inactivate the SIP1 protein include, but are not limited to, the expression of SIP1-specific antibodies. Preferably, the antibodies are specific for the zinc fingers and inhibit the binding of the SIP1 protein to the CACCT(G) promoter region of genes involved in differentiation and/or development, such as E-cadherin. Cells with high SIP1 expression may be specifically targeted using the CD44 marker, based on the correlation between SIP1 expression and the CD44high/CD24low expression.

Still another aspect of the invention is the use of the SIP1 gene and/or the SIP1 gene-encoded protein for the identification of breast cancer stem cell markers. Indeed, SIP1 expression coincides with the known breast cancer stem cell marker CD44high/CD24low. Moreover, expression of SIP1 is influencing expression of CD24. Therefore, comparison of expression data under conditions of high SIP1 expression and low SIP1 expression, as can be done, as a non-limiting example, by comparing microarray data of expression patterns under both conditions, will lead to other genes of which the up- or down-regulation may be useful as a cancer stem cell marker.

Still another aspect of the invention is the use of the SIP1 gene and/or the SIP1-encoded protein to determine breast cancer aggressiveness. Breast cancer aggressiveness is known to the person skilled in the art (see, amongst others, Henson and Patierno, 2004) and is associated, amongst others, to reduced survival, rapid tumor progression and tumor dedifferentiation. A higher number of breast cancer stem cells in the tumor, and/or a higher level of SIP1 expression in those cells, will increase the aggressiveness of the tumor. Therefore, SIP1 is a good marker for tumor prognosis and survival.

As a consequence, another aspect of the invention is the use of SIP1 gene repression and/or the use of SIP1 protein inactivation to lower breast cancer aggressiveness. Lower breast cancer aggressiveness can be assessed by a slower tumor growth after treatment, or even a decrease in tumor size.

Identification and targeting of the limited number of cancer stem cells in a whole population of tumor cells is expected to have major advantages in cancer treatment. Indeed, classical therapy is focusing on the killing of rapidly dividing cells, but does not eliminate the cancer stem cells, which are dormant most of the time. Specific detection and elimination of the cancer stem cells, before or after removal of the bulk (non-cancer stem cell) tumor cells, would allow a far more efficient cancer treatment.

### BRIEF DESCRIPTION OF THE FIGURES

FIG. 1: Expression of EMT-inducing transcription factors in MDAMB231. Quantitative RT-PCR for ZEB1/δEF1, ZEB2/SIP1, Slug and Snail in MDAMB231. Normalized expression levels are compared to the level of Snail, which was arbitrarily set at 1.

FIG. 2: RNAi-based knock-down of ZEB2/SIP1 in MDAMB231. Panel A) Quantitative RT-PCR for ZEB2/SIP1 and ZEB1/δEF1 in MDAMB231 cells stably transduced with empty vector (pLVTH) or vector containing a ZEB2/SIP1-directed short hairpin (SIP1kd). The ZEB2/SIP1 mRNA level is ±90% reduced while the ZEB1/δEF1 mRNA level is unaffected. Panel B) Alignment of the ZEB2/SIP1 siRNA sequence to a selected region of the ZEB2/SIP1 and ZEB1/δEF1 3′UTR mRNA sequences (SEQ ID NOS:12-14).

FIG. 3: ZEB2/SIP1 depletion enhances cell aggregation. Panel A) Phase contrast images of cultured MDAMB231 pLVTH and SIP1kd cells (upper panel). SIP1kd cells form bigger aggregates as shown in a slow aggregation assay (lower panel). Panel B) Quantitative RT-PCR shows an increased E-cadherin mRNA expression in the SIP1kd cells compared to pLVTH but this E-cadherin level is very moderate compared to E-cadherin expression in MDAMB231 cells with a ZEB1/δEF1 knock-down. Panel C) Western blot analysis for several cadherins, the tight junction protein ZO-1 and the desmosomal proteins desmogleïn and plakophilin 3. Only ZO-1 is significantly induced in the SIP1kd cells compared to control cells. Panel D) Immunofluorescence for β-catenin and ZO-1 shows a clear relocalization from the cytoplasm to the plasma membrane in the SIP1kd cells. The white bar represents 10 μm.

FIG. 4: ZEB2/SIP1 contributes to in vitro cell migration and invasion. Panel A) Wound closure assay performed on pLVTH- and SIP1kd-transduced MDAMB231 cells. Wounds made in confluent layers of SIP1kd cells close significantly slower compared to the pLVTH control cells. Bars represent the mean value of nine measuring points. Panel B) Matrigel invasion shows a ±40% reduction in invasive capacity of the SIP1kd cells compared to pLVTH cells. Three independent experiments were performed, the results of a representative experiment are shown here.

FIG. 5: ZEB2/SIP1 depletion has a moderate effect on in vitro cell proliferation. Cells were seeded and quantified every 24 hours over a time period of five days. The SIP1kd cells show a somewhat slower proliferation rate compared to the control cells.

FIG. 6: ZEB2/SIP1 knock-down drastically impairs soft agar colony formation. MDAMB231 pLVTH and SIP1kd cells were seeded at low density in soft agar and allowed to form colonies for 55 days. Colonies were then stained with crystal violet and photographed.

FIG. 7: Effect of ZEB2/SIP1 depletion on tumor growth of MDAMB231. Growth curve of MDAMB231 tumor xenografts. Tumors of mice injected with parental MDAMB231, pLVTH-transduced or SIP1kd-transduced cells were measured on the days indicated and tumor volumes were calculated.

FIG. 8: EGFP and ZEB2/SIP1 expression in MDAMB231 xenografts. Immunohistochemical detection of EGFP and ZEB2/SIP1 in dissected tumors of MDAMB231 pLVTH and SIP1kd cells. Most cells are EGFP-positive, while only a limited number of cells are positive for ZEB2/SIP1.

FIG. 9: Global altered gene expression patterns in ZEB2/SIP1 knock-down cells. Panel A) Schematic overview of some functional classes the differentially expressed genes can be classified in. Genes up-regulated as well as down-regulated in the MDAMB231 SIP1kd cells versus pLVTH control cells are taken into account. Genes involved in cell growth, proliferation, migration and cancer-related genes are particularly represented. Panel B) IPA-calculated functional network combining differentially expressed genes putatively involved in cell growth, proliferation and apoptosis. Panel C) IPA-calculated functional network of differentially expressed genes related to integrin function and signaling. Genes down-regulated in the SIP1kd cells are depicted in green, up-regulated genes are depicted in red.

FIG. 10: Colocalization of ZEB2/SIP1 with different stem cell markers, Immunofluorescent co-staining of breast stem cell markers ALDH1, CD44 and Nestin and ZEB2/SIP1 in breast epithelial cell lines MCF10A and MDAMB231.

FIG. 11: SIP1 expression is enhanced in invasive MDAMB231 cells.

FIG. 12: Expression analysis of a series of 56 primary human breast cancers. Relative SIP1/ZEB2 expression levels (average of ten samples with low expression set to 1) were depicted ranking high to low.

FIG. 13: Effect of SIP1 depletion on colonization of the mouse lung. Thirteen female nude mice were injected in the tail vein with control MDAMB231 cell lines. Ten mice were injected with the SIP1kd cell line. Panel A) Nine out of thirteen control (69%) mice and two out of ten SIP1kd-injected mice (20%) developed macroscopic lung nodules. Panel B) Representative pictures of murine lungs with metastasized MDAMB231 colonies from control and SIP1kd-injected mice.

FIG. 14: Hematoxilyn and eosin staining on lung sections of mice injected with control MDAMB231 cells and SIP1kd cells. The stainings parallel the presence or absence of tumors as seen macroscopically on the lung surfaces.

FIG. 15: SIP1 staining on lung sections of mice injected with control MDAMB231 cells and SIP1kd cells. Tumor outgrowths in the lungs of control-injected and SIP1kd-injected mice show a similar SIP1 expression pattern, resembling the SIP1 expression in the primary tumors as well.

FIG. 16: Study of breast cancer stem cell surface marker CD24. Panel A) FACS analysis of cell surface marker CD24 in MDAMB231 cells expressing constitutively or conditionally a SIP1/ZEB2 specific knock-down vector or a control vector. Panel B) QRT-PCR quantification of CD24 sorted cells described in Panel A for CD24 and SIP1/ZEB2 expression.

FIG. 17: Relationship between SIP1 and CD24 expression in breast cancer cell lines and breast tumors. Left panels: linear correlation; Right panels: Cell lines and tumors CD24 and corresponding SIP1 expression values ranked by increasing SIP1 expression. GEO accession numbers are indicated above each panels.

### DETAILED DESCRIPTION OF THE INVENTION

### Examples

### Materials and Methods to the Examples

### Construction and Transduction of Short Hairpin-Containing Lentiviral Vectors.

A ZEB2/SIP1-specific siRNA sequence was designed using selection criteria as described (Brummelkamp et al., 2002; Ui-TEi et al., 2004). A double PCR approach was used to create an shRNA expression cassette, which was subsequently cloned in the lentiviral pLVTH vector (Wiznerowicz and Trono, 2003) using EcoRI and ClaI restriction sites. The primers for the first PCR were 5′-CTGCAGGAATTCGAACGCTGACGTCATCAA-3′ (SEQ ID NO:1) and 5′-AAATCTCTTGAATTTAACAATACCCAGCTCCGGGGATCTGTGGTCTCATACAGAA CTTATAA-3′ (SEQ ID NO:2). This PCR product was a template for a second PCR reaction with the same forward primer and the reverse primer 5′-CCATCGATAAGCTTTTTTTCCAAAAAAGGAGCTGGGTATTGTTAAATCTCTTGAAT TTA-3′ (SEQ ID NO:3). Lentivirus production and transduction were performed as described below:

For lentivirus production, 1.2 million cells of the packaging cell line HEK293T were seeded in a 25-cm² flask. After 24 hours, 3 μg of the modified pLVTH vector, 3 μg of the packaging plasmid pCMVdR8.91 (Trono) and 1.5 μg of the envelope plasmid pMD2G-VSVG (Trono) were first precipitated together and then transfected into the HEK293T cells using a standard calcium phosphate precipitation method. After eight hours, the transfection medium was removed and the cells were incubated for 48 hours in 4 ml of fresh culture medium. The virus-containing medium was then harvested and filtered through a 0.45 μm low protein binding filter unit (Millipore). Aliquots were stored at −70° C. Transduction was performed by mixing 50,000 cells with 200 μl viral supernatant in a 96-well plate. These mixtures were centrifuged for 1.5 hours at 32° C. and 1500 rpm and subsequently incubated at 37° C. After 24 hours, the medium was replaced by fresh culture medium or fresh virus-containing medium in case a second round of transduction was performed. Transduction efficiencies were determined by measuring EGFP expression by flow cytometry (FACSCalibur; Becton Dickinson). If necessary, cells were sorted to obtain populations with more than 90% EGFP-positive cells.

### Immunocytochemistry

Fixation and immunofluorescence were performed following standard procedures (Van Hengel et al., 1999).

### SDS-PAGE and Immunoblotting

These techniques were performed as described (Vandewalle et al., 2005).

### Antibodies

The primary rabbit anti-ZO1 antibody (1/100, Zymed) and the rabbit anti-β-catenin antibody (1/1000, Sigma) were used for immunocytochemistry. The mouse anti-ZEB2/SIP1 antibody (1/100) and rabbit anti-EGFP antibody (1/200, Molecular Probes) were used for immunohistochemistry on paraffin-embedded sections. The mouse HECD1 antibody anti-E-cadherin (1/100, Takara), the mouse anti-N-cadherin antibody (1/1000, Transduction), the mouse anti-P-cadherin antibody (1/250, Transduction), the mouse Nestin (1/200, Chemicon), the mouse CD44 (1/200, BD Pharmingen), the mouse ALDH1 (1/100, BD Transduction), the antimouse Alexafluor 488 (1/500, Molecular Probes A11029), the rabbit Pan-cadherin antibody (1/300, Sigma), the rabbit anti-ZO1 antibody (1/100, Zymed), the mouse anti-plakophilin-3 antibody (1/1000, 23E3/4) and the mouse anti-desmoglein 3.10 antibody (1/10, Progen) were used for immunoblotting.

### Real-Time Quantitative RT-PCR

qRT-PCR was carried out as described in sections VI.3. and VI.4., as were the primer and probe sequences for human ZEB2/SIP1, E-cadherin and N-cadherin. Sequences of primers for ZEB1/δEF1 were 5′-TGTTACCAGGGAGGAGCAGTG-3′ (SEQ ID NO:4) and 5′-TCTTGCCCTTCCTTTCTGTCA-3′ (SEQ ID NO:5). The primers and probe for Snail were 5′-CAGGACTCTAATCCAGAGTTTACCTTC-3′ (SEQ ID NO:6), 5′-GGGATGGCTGCCAGCA-3′ (SEQ ID NO:7) and 5′-FAM-AGCAGCCCTACGACCAGGCCCA-TAMRA-3′ (SEQ ID NO:8). The primers and probe for Slug were 5′-GCCAAACTACAGCGAACTGGA-3′ (SEQ ID NO:9), 5′-TGTGGTATGACAGGCATGGAG-3′ (SEQ ID NO:10) and 5′-FAM-CACATACAGTGATTATTTCCCCGTATCTCTA-TAMRA-3′ (SEQ ID NO:11).

### Slow Aggregation Assay

Bacto-agar (Difco) was dissolved in Ringer's salt solution (2% w/v) and boiled to sterilize. Flat 96-well plates were coated with 50 μl dissolved agar and cooled for at least one hour. Subsequently, 20,000 cells/200 μl/well were seeded on top of the agar. Twenty-four hours later, aggregates were analyzed by phase contrast microscopy (Olympus) and photographed. Ringer's salt solution contains 8.6% NaCl, 0.3% CaCl₂.2H₂O and 0.3% KCl in distilled water and is set to pH 7.4 with NaOH.

### Wound Closure Assay

Cells were seeded in duplicate in 60 mm tissue culture dishes at a density of 5×10⁵. Three incisions per well were made in the confluent culture 24 hours later, using a 300 μl pipette tip. After replacing the medium to remove floating cells, every wound was measured at three different, clearly marked points by phase contrast microscopy (Olympus). The cells were then incubated at 37° C. for four hours, before the wounds were measured again. The distance of migration was calculated by subtracting the measured wound values and averaged for the nine measuring points per dish.

### Matrigel Invasion Assay

This assay was performed as described by the protocol provided with the QCM 24-well cell invasion assay, fluorometric kit (Chemicon).

### Affymetrix GeneChip Analysis

The microarray experiment was performed by the VIB MicroArray facility (MAF), and included probe labeling and hybridization on Affymetrix GeneChip (Human Genome U133 Plus 2.0) and subsequent data acquisition and processing. Expression values were downloaded from studies profiled with the Affymetrix GeneChip Human Genome U133 Plus 2.0 cDNA Array deposited in the NCBI GEO database. For each study, the expression values for the probes sets of interest were extracted for each sample and normalized by measuring the distance between each probe expression value and the corresponding lowest value for the same probe in the same study. This distance was then expressed relative to the standard deviation of the mean of the probe expression values of the analyzed study. A gene was scored as downregulated if AvRatio<0.5. A gene was scored as upregulated if AvRatio>2. Expression values of the probes set 203603_s_at (SIP1/ZEB2) were compared to the expression value of other probes by linear correlation. Linear correlation Pearson coefficient between the expression values for the probe 203603_s_at (SIP1/ZEB2) and the expression values of the indicated probes computed for each study are illustrated in Table 2.

### In Vitro Proliferation Assay

Cells were seeded in multiple flat 96-well plates in a final concentration of 7000 cells/200 μl/well. Six replicate wells were made of each condition. Every 24 hours, one 96-well plate was fixed by adding 50 μl TCA (Trichloric acid) (Reidel-de-Haen) directly to the culture medium before incubating at 4° C. for one hour. The wells were then rinsed five times with tap water and allowed to air dry thoroughly. The cells were stained with 100 μl of 0.4% SRB in 1% acetic acid for 30 minutes and then rinsed four times with 1% glacial acetic acid. When dry, 200 μl/well of 10 mM Tris buffer, pH 10.5 was added to release the bound dye. After mixing, the plates were measured in a multi-well reader (Benchmark, BioRad) at 490 nm.

### Colony Formation in Soft Agar

Petri dishes (60 mm diameter) were coated with 0.5% Bacto agar (Difco) in growth medium. A suspension of 1×10⁴ cells in growth medium containing 0.35% Noble agar (Difco) was seeded on top of the agar coating and then covered with growth medium. Fresh medium was applied weekly. Cell colonies were allowed to form over a time period of 55 days after which they were stained for one hour with 0.005% crystal violet (Sigma) in PBS and photographed.

### Orthotopic Injection in Nude Mice

A suspension of 1×10⁶ cells in a 100 μl mixture of culture medium and matrigel (BD Biosciences) (1:1) of each cell line was injected in the mammary fat pad of six-week-old female Rj:NMRI-nu (nu/nu) mice (Janvier). Tumors were measured weekly using a calliper and tumor volumes were measured using the formula (W²×L×π)/6. Mice were sacrificed when the largest tumors reached a 15-20 mm diameter. Tumors were then collected and subjected to immunohistochemical analysis.

### Immunohistochemistry on Paraffin-Embedded Tissue Sections

Mouse tissues were fixed in 2% paraformaldehyde, dehydrated and paraffin-embedded. For staining, paraffin was removed and sections were rehydrated. Endogenous peroxidase was blocked with a 1/100 dilution of H₂O₂ in methanol. Antigen retrieval was carried out in a pressure cooker (2100 Retriever) while soaking the sections in an antibody-dependent buffer. For ZEB2/SIP1 and cyclinD1, a citric acid buffer pH6 was used (1.8 ml 1 M citric acid and 8.2 ml Na-citrate in 1 liter deionized water). Aspecific binding sites were blocked with 3% goat serum in PBS+1% BSA. Incubation with the primary antibody (diluted in PBS/BSA) was done overnight at 4° C. Incubation with the biotinylated secondary antibody (1/400, Dako) was carried out for 30 minutes at room temperature. This was followed by a 20-minute incubation with the StreptABComplex/HRP (Dako) according to the manufacturer's protocol. After washing, the sections were treated with DAB (BioGenex) following the standard procedure. Staining was stopped by rinsing with tap water, after a brown color had appeared. Hematoxylin staining was done following the standard protocol. Finally, the sections were dehydrated and imbedded in pertex (HistoLab).

### Tail Vein Injection in Nude Mice

A suspension of 2×10⁶ tumor cells in a 150 μl physiologic salt solution was injected into the tail vein of six-week-old female Rj:NMRI-nu (nu/nu) mice using a 26-gauge needle. Mice were monitored periodically and sacrificed 22 weeks later. Lungs were dissected from the thoracic cage and carefully examined for the presence of micro- and macrometastatic lesions.

### Flow Cytometric Analysis

By using a Calibur flow cytometer (Becton Dickinson and Company, San José, Calif.) the expression of CD24 and CD44 was distinctly evaluated on MDAMB231 cells with conditional expression of a SIP1kd expression plasmid. The antibodies used were PE-coupled anti-CD24 and APC-coupled anti-CD44 obtained from BD Pharmingen. Briefly, cells were trypsinized and 10⁶ cells were stained in suspension for one hour with a 1/20 dilution of the antibodies in PBS+0.5% BSA before being FACS-analyzed.

### SIP1 Expression Analysis in Human Primary Breast Cancers

cDNA synthesis on RNA samples was performed on 2.5 μg total RNA using the Iscript cDNA synthesis kit (Bio-Rad). Subsequently, qPCR on the LC480 (Roche) was done for SIP1 and different reference genes (Vandesompele et al., 2002) using LC 480 Sybr Green I master kit (Roche), Fast SYBR master mix kit (Applied Biosystems) and Taqman fast univ. PCR Mastermix (Applied Biosystems). Using GeNorm (Vandesompele et al., 2002), we determined the most accurate set of reference genes for normalization (HMBS, SDHA, TBP and UBC). The average threshold cycle of triplicate reactions was used for all subsequent calculations using the delta Ct method. Relative SIP1 expression levels (average of ten samples with low expression set to 1) were depicted ranking high to low.

### Example 1

### MDAMB231 Expresses an Array of EMT-Inducing Transcription Factors

As mentioned, MDAMB231 breast cancer cells show many features of mesenchymal cells including loss of E-cadherin expression. As E-cadherin down-regulation is often enforced by the action of transcriptional repressors, we examined the expression status of the E-cadherin repressors ZEB1/δEF1, ZEB2/SIP1, Slug, Snail and Twist in MDAMB231 by quantitative RT-PCR. ZEB1/δEF1 shows the highest expression level, followed by Slug while ZEB2/SIP1 and Snail are only moderately expressed (FIG. 1). The cells were negative for Twist expression.

Selective knock-down of ZEB1/δEF1 in MDAMB231 leads to re-acquired epithelial-specific features concomitant with re-expression of E-cadherin and other epithelial-specific proteins (Aigner et al., 2007). We wanted to investigate whether the moderate ZEB2/SIP1 expression in this cell line has a functional contribution to the dedifferentiated state of these cells. We therefore created an RNAi-based stable ZEB2/SIP1 knock-down in the MDAMB231 cell line and analyzed the functional and molecular effects.

### Example 2

### Reduced ZEB2/SIP1 Expression Leads to Enhanced Cell-Cell Adhesion

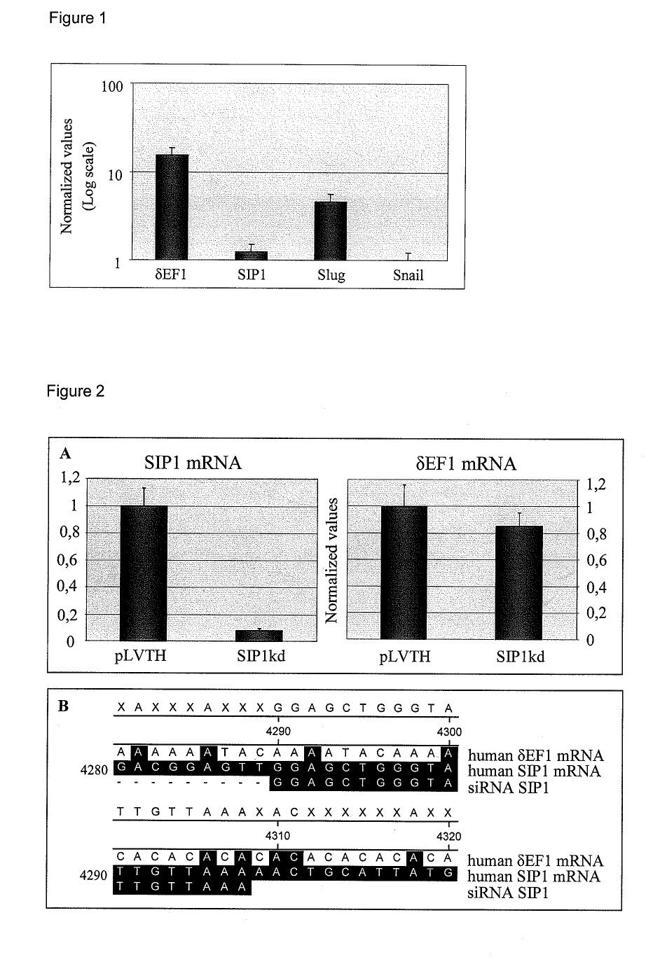

A lentiviral vector (pLVTH) (Wiznerowicz and Trono, 2003) containing a short hairpin RNA sequence designed to specifically target ZEB2/SIP1 mRNA, was stably transduced in MDAMB231, as was the empty vector. Quantitative RT-PCR showed a ±90% reduction of ZEB2/SIP1 mRNA expression in the knock-down cells (SIP1kd) as compared to the empty vector-transduced control cells (pLVTH). No down-regulation of the expression level of the closely related family member of ZEB2/SIP1, ZEB1/δEF1 could be detected (FIG. 2, Panel A). This is concomitant with the low sequence similarity of the 3′UTR sequences of ZEB2/SIP1 and ZEB1/δEF1 to which the ZEB2/SIP1 siRNA is targeted (FIG. 2, Panel B).

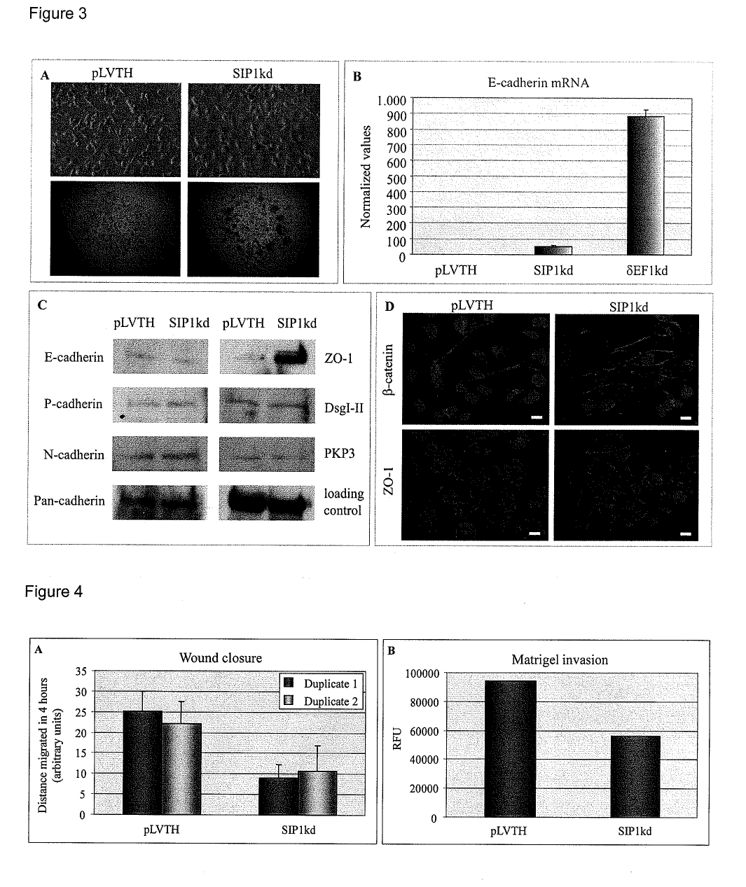

As MDAMB231 SIP1kd cells showed a less pronounced mesenchymal morphology and exhibited closer contacts with their neighboring cells, as compared to the pLVTH control cells (FIG. 3, Panel A), we examined whether the ZEB2/SIP1 knock-down has an effect on cell-cell adhesion. We therefore performed a slow aggregation assay that showed a somewhat stronger clustering of the SIP1kd cells compared to the pLVTH cells (FIG. 3, Panel A). As ZEB1/δEF1 depletion in MDAMB231 cells strongly induces E-cadherin expression (Eger et al., 2005 and FIG. 3, Panel B), we checked whether re-expression of E-cadherin is at the basis of the cell aggregation in the ZEB2/SIP1 knock-down cells as well. Quantitative RT-PCR and Western blot analysis, however, showed no significant E-cadherin up-regulation as compared to ZEB1/δEF1 knock-down cells, which strongly re-express E-cadherin (FIG. 3, Panels B and C). Most likely, the remaining high expression level of ZEB1/δEF1 and other E-cadherin repressors like Slug in the SIP1kd cells is responsible for the lack of detectable E-cadherin expression. Interestingly, immunofluorescence analysis for β-catenin, a cytoplasmic cadherin-binding partner, showed a marked relocalization from the cytoplasm to the plasma membrane (FIG. 3, Panel D). To check whether another cadherin, besides E-cadherin, could therefore be responsible for the enhanced cell-cell adhesion, Western blot detection for P-cadherin, N-cadherin and Pan-cadherin was performed. The Pan-cadherin antibody recognizes a conserved C-terminal sequence present in most classical cadherin family members (Geiger et al., 1990). Although cadherins are present, no significant increase in protein expression could, however, be detected (FIG. 3, Panel C).

Next, protein expression of components of other junctional complexes, including the tight junctions and the desmosomes, was examined. No altered expression pattern could be detected for plakophilin 3 and desmoglein I and II (FIG. 3, Panel C). However, a strong induction of ZO-1 protein and relocalization to the plasma membrane was seen in the ZEB2/SIP1 knock-down cells (FIG. 3, Panels C and D). ZO-1 is a cytoplasmic scaffolding protein linking the transmembrane tight junction proteins occludin and claudin to the cytoskeleton, but has also been shown to localize to adherens junctions through direct binding to a-catenin (Itoh et al., 1997; Muller et al., 2005). Thus, ZO-1 possibly forms a link between the adherens and the tight junction. Even though so far we were unable to show induced cadherin expression, this does not exclude the possible involvement of a cadherin that we did not detect with the Pan-cadherin antibody or the relocalization of a cadherin to the plasma membrane. Alternatively, involvement of the tight junctions cannot be excluded either, as induced expression of occludin or claudin could result in recruitment of ZO-1 and its binding partners to the tight junctions. Indeed, it was recently reported that adherens junction formation is not necessarily required for tight junction assembly as HepG2-AJ⁻ cells that are unable to form adherens junctions slowly form functional tight junctions that mediate cell-to-cell interactions and cell polarity (Thread et al., 2007).

Further experiments are thus required to identify the specific molecules within the tight junctions, adherens junctions and maybe desmosomes, responsible for the enhanced aggregation.

### Example 3

### ZEB2/SIP1 Contributes to Tumor Cell Migration and Invasion In Vitro

MDAMB231 cells are highly motile and to investigate the effect of ZEB2/SIP1 depletion on directional cell migration, we performed in vitro wound closure assays. In a time period of four hours, the SIP1kd-transduced cells had only migrated half the distance of the pLVTH-transduced cells (FIG. 4, Panel A). Knock-down of ZEB2/SIP1 thus impairs in vitro cell migration. These data are in agreement with the results obtained in another breast cell line, MCF10A, in which reduced ZEB2/SIP1 expression coincides with down-regulated vimentin expression and diminished migratory capacities (Bindels et al., 2006).

We next performed matrigel invasion assays to determine whether ZEB2/SIP1 knock-down also affects the in vitro invasive behavior of the MDAMB231 cells. Both pLVTH-transduced and SIP1kd-transduced cells were seeded in serum-free conditions on top of matrigel-coated transwell filters and allowed to migrate towards serum-containing medium for 24 hours. The invaded cells were then fluorescently labeled and quantified. A reduced invasion of approximately 40% could be detected in the SIP1kd-transduced cells as compared to the pLVTH control cells (FIG. 4, Panel B). The above-described data indicate that ZEB2/SIP1, despite its low expression level in MDAMB231 cells, contributes to both the migratory and invasive capacities of these cells in vitro.

### Example 4

### Impact of ZEB2/SIP1 Knock-Down on In Vitro and In Vivo Growth

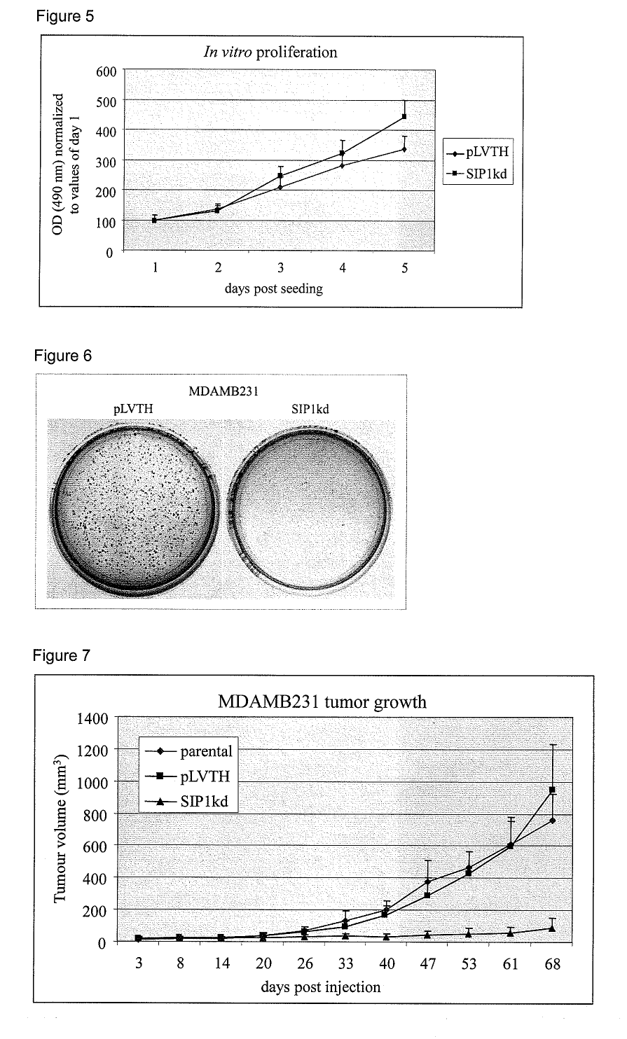

To analyze the effects of the ZEB2/SIP1 knock-down on cell proliferation in vitro, we seeded equal amounts of cells in several 96-well culture plates. Each day, during five consecutive days, the cells in one plate were fixed and quantified in a multi-well plate reader after staining with SRB. The growth curves of the ZEB2/SIP1 knock-down cells show a moderate decrease in their cell growth compared to the pLVTH cells (FIG. 5).

We next evaluated the effect of ZEB2/SIP1 depletion on anchorage-independent growth. We therefore seeded MDAMB231 pLVTH and SIP1kd cells in soft agar and allowed them to form colonies over the next two months. As shown in FIG. 6, the colony-forming activities of the SIP1kd cells are drastically reduced compared to the pLVTH cells.

As MDAMB231 cells are tumorigenic when injected in nude mice, we wanted to determine whether knocking-down ZEB2/SIP1 in MDAMB231 has an impact on the in vivo tumorigenicity and tumor progression. Parental MDAMB231, pLVTH-transduced and SIP1kd-transduced cells were mixed with matrigel and injected in the mammary fat pad of immunocompromised female nude mice. We chose to implant the cells orthotopically since the take rate and the progression of many tumors is highly dependent on the specificity of tumor-host and tumor-stromal interactions. The parental MDAMB231 cells were injected in a total number of six animals, the pLVTH and SIP1kd cells were each injected in twelve mice. Tumors were measured weekly and volumes were calculated. All the animals injected with parental MDAMB231 and pLVTH-transduced cells formed exponentially growing tumors after a latency period of approximately three weeks. Tumors of mice injected with SIP1kd-transduced cells, however, remained much smaller in size (FIG. 7). Mice were sacrificed once the largest tumors had reached the maximum allowed volume prescribed by the ethical guidelines. The tumors were removed, fixed and paraffin-embedded for further histological analysis. No macroscopic signs of local invasion or metastasis were present.

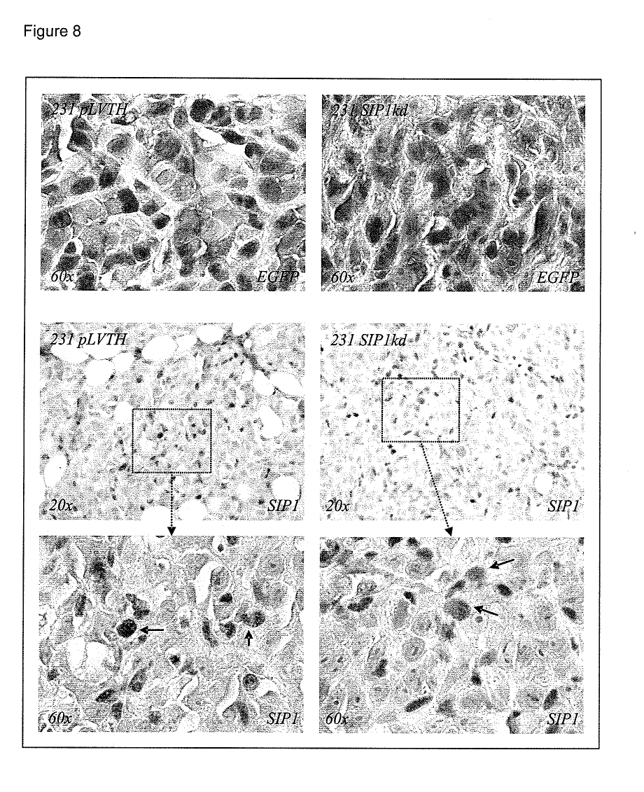

As the pLVTH- and SIP1kd-transduced cells are EGFP-labeled, we performed an immunohistochemical analysis with an EGFP-specific antibody to show the presence of the transduced MDAMB231 cells in the tumor sections (FIG. 8). As expected, most of the cells were EGFP-positive showing the presence of the transduced MDAMB231 cells in these tumor sections.

We next wanted to assess whether ZEB2/SIP1 can be detected in these sections, using our ZEB2/SIP1-specific antibody (7F7). The staining revealed the presence of solitary ZEB2/SIP1-positive cells dispersed throughout the tumor. These cells could be ZEB2/SIP1-positive mouse stromal cells intermingled with the tumor cells. Alternatively, as MDAMB231 is a very heterogeneous cell population, it is possible that only a few cells within this population express ZEB2/SIP1 protein at a detectable level. A way to discriminate between human and mouse cells in these tumor sections is to perform an immunohistochemical analysis with an antibody specifically labeling human or mouse cells, respectively. Major histocompatibility complex class I proteins are expressed on the cell surface of all nucleated cells and due to the high level of allelic diversity within this gene family, they are particularly good candidates to specifically mark human or mouse cells, respectively. Thus, co-staining for ZEB2/SIP1 (nuclear) together with a mouse-specific or human-specific MHC antibody (membranous) should allow us to identify the human or murine nature of the ZEB2/SIP1-positive cells. Staining of the sections with a human-specific MHC class I antibody has been performed. However, as the staining on the cell surface of the abundantly present human cells also lines solitary, neighboring negative (murine) cells, this approach appears to be difficult to distinguish the human and mouse cells. A better strategy would therefore involve the use of a mouse-specific antibody as far less mouse cells are expected. However, most commercially available antibodies are used for FACS analysis only and are not proposed to work on paraffin-embedded tissue sections. Nevertheless, mouse-specific antibodies will be tested.

### Example 5

### Altered Gene Expression Patterns in MDAMB231 ZEB2/SIP1 Knock-Down Cells

In order to screen for molecular changes that coincide with knock-down of ZEB2/SIP1 in MDAMB231, we performed a transcriptome-wide differential gene expression experiment using Affymetrix GeneChip arrays. To this end, cDNA of MDAMB231 pLVTH cells was compared to SIP1kd cDNA.

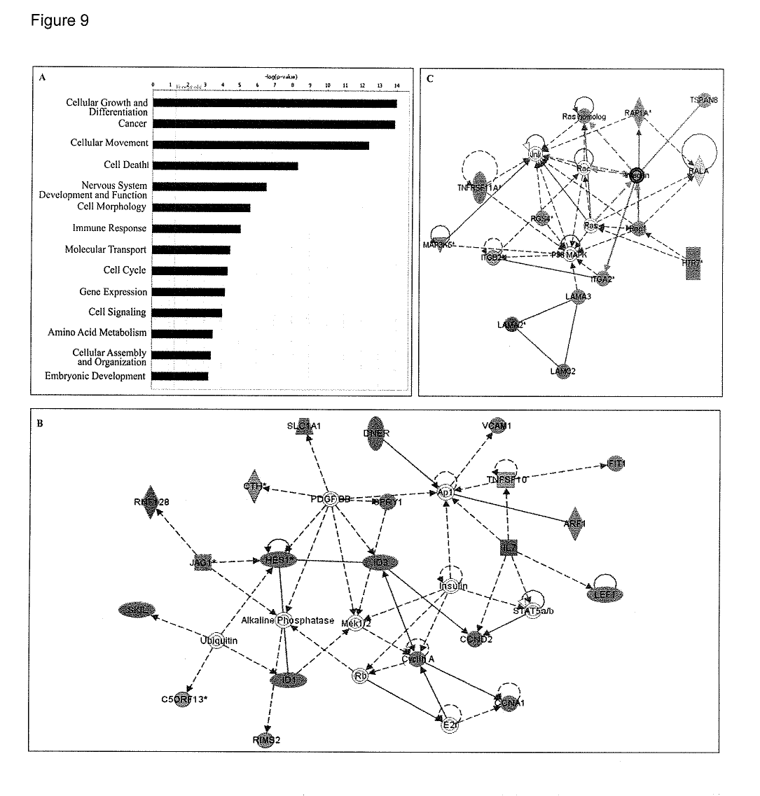

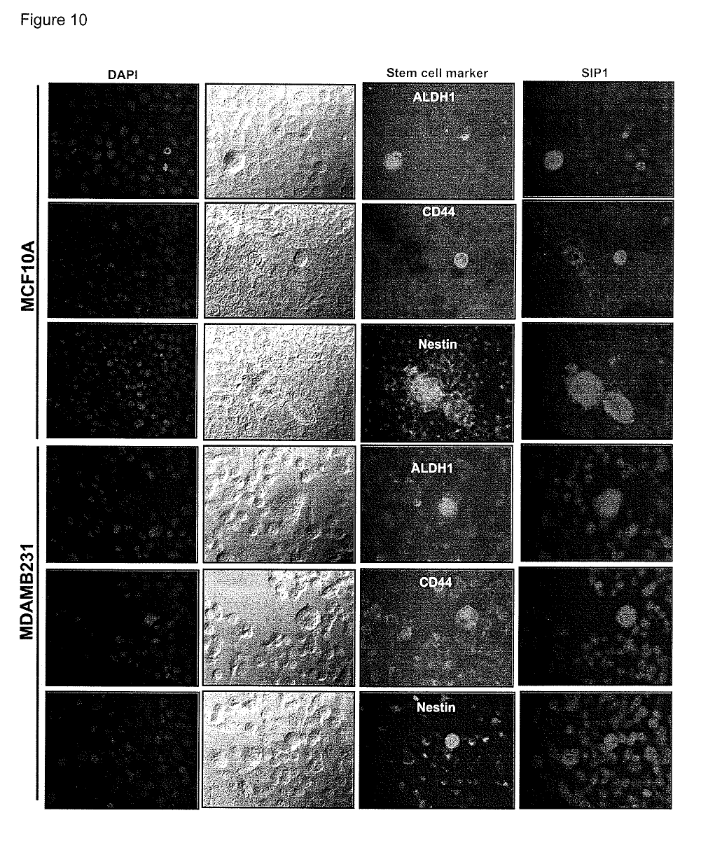

When we analyzed the pLVTH and SIP1kd expression profiles, 502 genes appeared to be differentially expressed with 225 genes showing a more than two-fold up-regulated expression in the SIP1kd and 277 genes showing a more than two-fold down-regulation. The web-based tools Onto-Express (on the worldwide web at vortex.cs.wayne.edu:8080) and Ingenuity Pathways Analysis (IPA, on the worldwide web at ingenuity.com) were then used to annotate and cluster these genes into functional classes and putative networks. When making a classification according to biological function, genes acting in many different cellular and molecular pathways as well as disease-related genes are represented (FIG. 9, Panel A). Genes involved in cell growth and cell movement, as well as cancer-related genes, are particularly present. Striking is the absence of differentially expressed major cell-cell adhesion molecules, normally strongly affected by ZEB2/SIP1. The remaining high ZEB1/δEF1 expression level is most likely responsible for the continued repression of these genes. The enhanced cell aggregation of the SIP1kd cells is then probably the sum of subtle gene expression changes that were not detected in our current microarray analysis.

An interesting tool within the Ingenuity software converts large data sets into networks containing direct and indirect relationships between genes based on known interactions in the literature. One should be aware however that even though potentially interesting links are made by these software, the data need critical evaluation, combining literature and experimental validation. FIG. 9, Panel B, shows an association between several differentially expressed signaling components involved in growth, proliferation and apoptosis. Expression of several genes regulated by or implicated in Notch-signaling (JAG1, HES1, DNER), TGF-β-signaling (ID1, ID3, SKIL, C5ORF13), PDGF-signaling (ID3, HES1, CTH, SPRY1, SLC1A1) and Wnt-signaling (LEF1) appears to be affected in the SIP1kd-transduced cells, which may severely impact tumor cell growth in an autocrine, as well as paracrine, fashion. Also particularly interesting is the down-regulation of the inhibitors of differentiation (ID1 and ID3) as their expression was shown to be required for tumor initiation of both primary breast tumors as well as metastatic colonization in the lungs (Gupta et al., 2007). In addition, reduced expression of cyclin genes (cyclin A1 and D2) may slow down the cell cycle. Alternatively, the up-regulated expression of the pro-apoptotic cytokine TNFSF10 (TRAIL) in the SIP1kd cells may enhance tumor cell death (Schaefer et al., 2007). Another interesting network shows a simultaneous down-regulation of interconnected components of integrin signaling as well as ECM proteins (laminin-α2, -α3 and -γ2), which could partly explain the reduced migration seen in the ZEB2/SIP1 knock-down cells (FIG. 9, Panel C).

This preliminary analysis suggests that putatively interesting links may exist between the observed in vitro and in vivo functional effects of the ZEB2/SIP1 knock-down and differential gene expression patterns and more interesting pathways will undoubtedly be unraveled after extensive exploration of the dataset using several available web-based tools. However, the differential expression of these genes, as well as their possible involvement in ZEB2/SIP1-dependent functional consequences, needs to be confirmed and validated, as further explained in the discussion section. Another appealing prospect is the clustering of this dataset with the earlier-described dataset of differentially expressed genes after ZEB2/SIP1 overexpression. Genes that are differentially expressed in both the forward and the reverse experiment may define a relevant set of ZEB2/SIP1-target genes, independent of cell type and context.

### Example 6

### ZEB2/SIP1 Co-Staining with Stem Cell Markers

Tumor-initiating cells or cancer stem cells can be distinguished from the non-tumor-initiating cancer cells based on well-accepted stem cell marker expression like, for instance, CD44, which is a cell surface marker and nestin, which is a filamentous protein and neural stem cell marker. ALDH1 is a cytosolic isoenzyme responsible for oxidizing intracellular aldhydes, responsible for oxidizing intracellular aldehydes, leading to the oxidation of retinol to retinoic acid in early stem cell differentiation. Immunofluorescent co-staining of MCF10A and MDAMB231 human breast epithelial cell lines with CD44, ALDH1 and nestin shows that rare cells are both positive for these cancer stem cell markers and ZEB2/SIP1.

### Example 7

### SIP1 Expression Analysis in Invasive MDAMB231 Cells

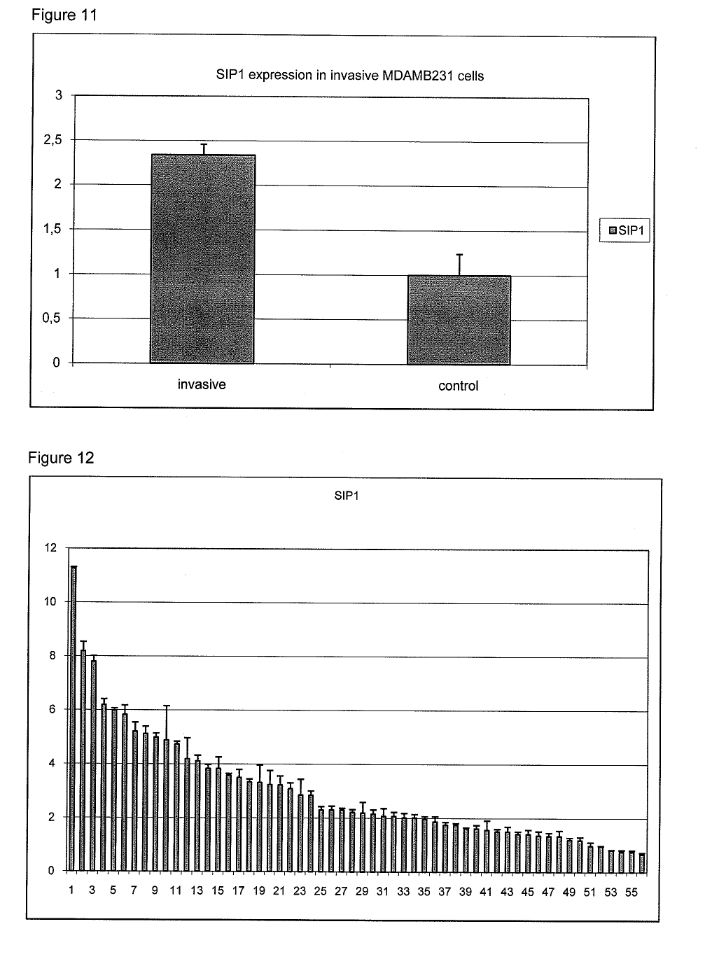

A matrigel invasion assay for MDAMB231 cells was performed as described by the protocol provided with the QCM 24-well cell invasion assay fluorometric kit (Chemicon), with the exception that invasive cells were not fluorescently analyzed but lysed for RNA preparation and further analysis by qRT-PCR. The control setting contained the complete MDAMB231 population, seeded on plastic and analyzed in parallel. cDNA synthesis and qPCR for SIP1 and two housekeeping genes (TBP and HMBS) was performed as described in X3. Invasive MDAMB231 cells showed higher SIP1 expression in comparison with the complete MDAMB231 cells (FIG. 11). Furthermore, we analyzed with QRT-PCR a large series of primary human breast cancers and confirmed the presence of SIP1 in a wide range of expression levels (FIG. 12).

### Example 8

### SIP1/ZEB2 Knockdown Block Lung Metastasis Formation

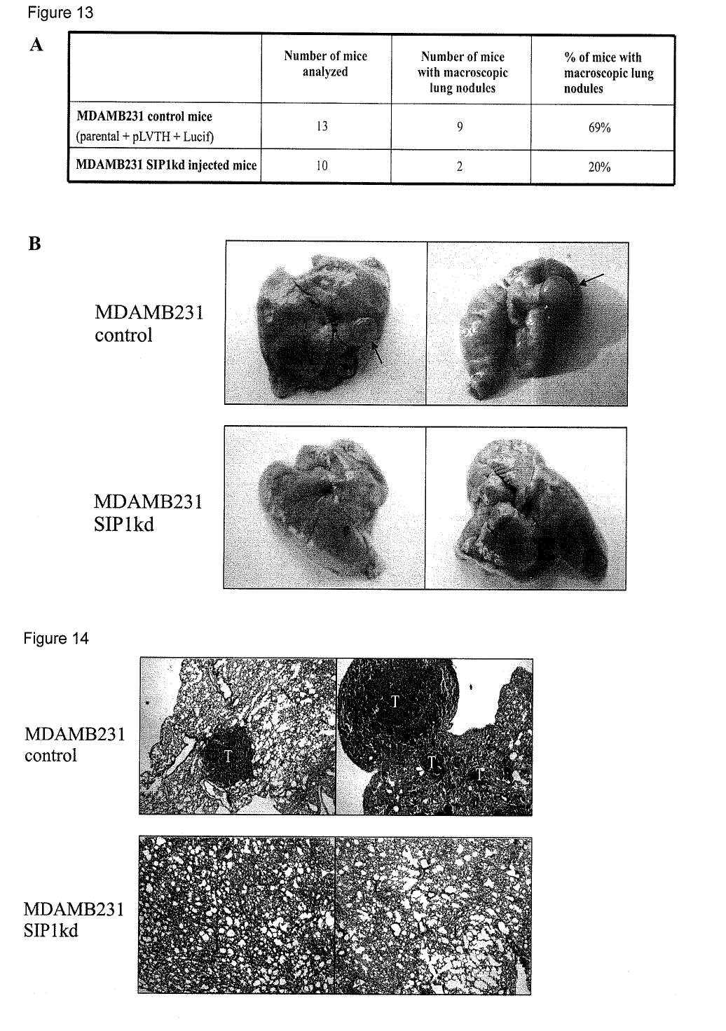

The drastic difference in tumor-forming capacity between the SIP1kd-transduced cells and control cells makes it difficult to monitor and compare the subsequent steps in tumor progression, including invasion and metastasis. Injection in the lateral tail vein, often termed “experimental metastasis,” is an alternative injection route often used to study the ability of tumor cells to home to and colonize the lungs, mimicking late malignant steps as survival in the circulation, extravasation and colonization, thereby bypassing the step of primary tumor formation and subsequent local invasion and intravasation. A total number of 13 mice served as a control setting. These mice were either injected with parental MDAMB231 cells (two mice), pLVTH-transduced cells (seven mice) or cells transduced with a luciferase-expressing vector (four mice). The SIP1kd cells were injected in the tail veins of ten mice. After a time period of 22 weeks, nine out of thirteen control mice (69%) had developed macroscopically visible lung nodules, although this was only the case for two out of ten (20%) of the mice injected with SIP1kd cells (FIG. 13, Panel A). The presence or absence of lung metastases was further documented by hematoxilyn/eosin staining of lung sections (FIGS. 13, Panel B, and 14). These stainings paralleled the number and size of the tumors macroscopically detected on the lung surfaces. These data indicate that SIP1 expression in MDAMB231 is not only critical for primary tumor growth, but also drastically affects the potential of these cells to colonize lung tissue and form secondary tumor outgrowths there.

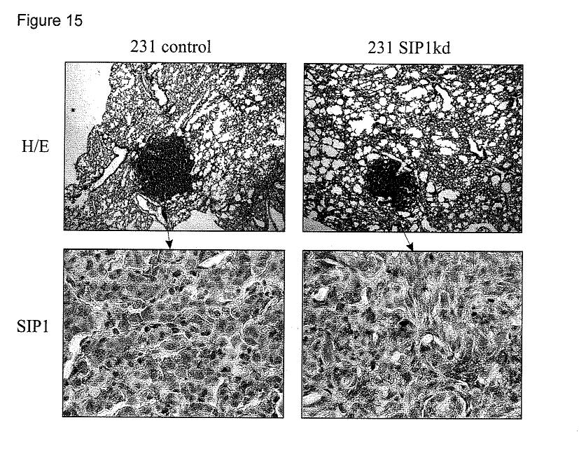

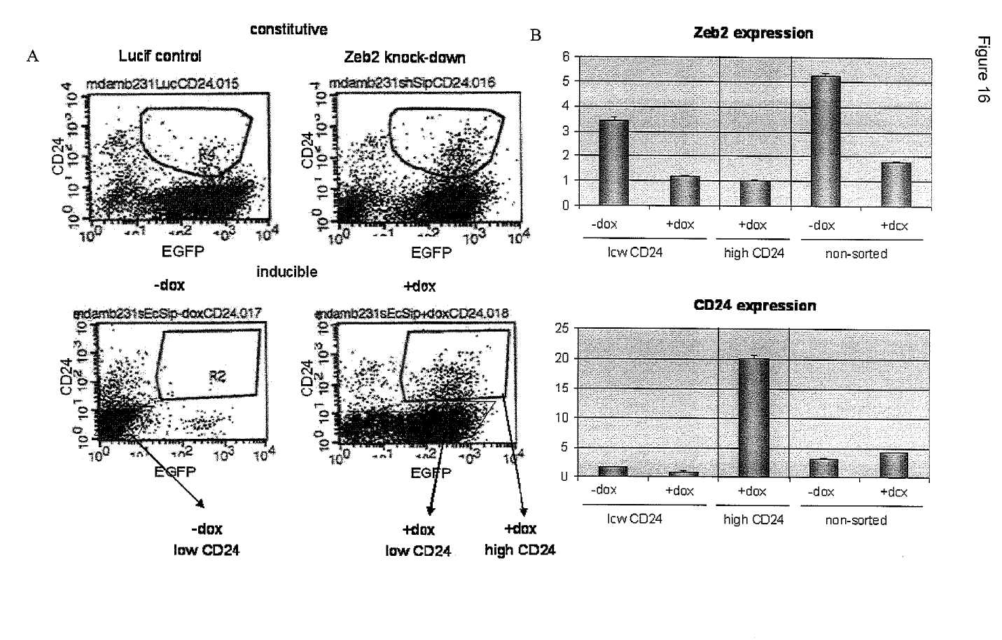

As mentioned above, in two out of ten mice injected with the SIP1kd cells, lung tumors were able to develop. This may not be that surprising as metastasis formation was monitored over a substantially long time span (22 weeks). During this time, it is possible that SIP1kd cells have lost their knock-down capacity or are simply outgrown by remaining SIP1-expressing cells in the population. Indeed, immunohistochemical analysis for SIP1 showed a similar SIP1 expression pattern in the lung tumor sections of control and SIP1kd-injected mice (FIG. 15). Furthermore, this SIP1 expression pattern highly resembles the SIP1 expression in the primary tumors, emphasizing the possibility of tumor formation by a limited number of tumor-initiating SIP1-positive cells.

### Example 9

### SIP1/ZEB2 Modulates the Breast Cancer Stem Cell Surface Phenotype CD44^{high}/CD24^{low}

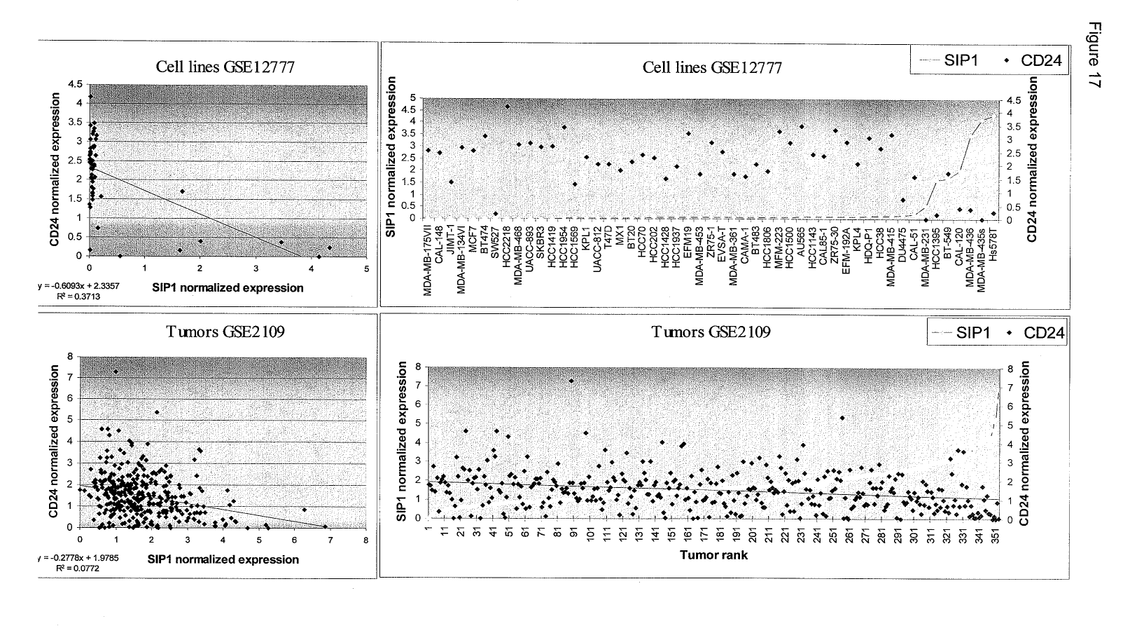

A small number of cells within a tumor have properties that resemble those of stem cells. The first tumor-initiating cells in a solid tumor were isolated from breast cancer. These cells efficiently formed anchorage-independent mammospheres. Mammospheres are known to be derived from mammary epithelial stem cells. It has been shown that a single mammosphere can give rise to an entire mammary ductal tree when implanted in mice. When isolated from primary breast cancers or from clinically apparent metastatic lesions, the cell surface phenotype CD44^{high}/CD24^{low} cells have cancer stem cell activity as indicated by the fact that they can differentiate and are highly tumorigenic when injected into immunodeficient mice (Al-Hajj et al., 2003). As few as 200 cells displaying this phenotype form tumors in immunodefficient mice, whereas 200,000 cells that do not display these markers fail to form tumors. Fitting with the stem cell model, the CD44^{high}/CD24^{low} cells generate tumors that recapitulate the phenotypic heterogeneity of the original tumor. These cancer stem cells compose 1% to 10% or primary or metastatic lesions. To determine whether these well-established breast cancer stem cell markers are changing upon ZEB2 expression modulation, we evaluated the expression of CD44 and CD24 in the MDAMB231 cells by flow cytometry. The large majority of these cells stained positive for CD44 and negative for CD24. However, upon conditional knock-down of SIP1, a subpopulation accounting for 3% to 5% of the total cell number became highly CD24 positive (FIG. 16). As in our hands, only a small percentage of cancer cells of the MDAMB231 xenografts is clearly SIP1/ZEB2-positive. One could speculate that these are the cancer stem cells that are capable of driving tumor colonization. To further support the relation between SIP1/ZEB2 and CD24 expression in human breast cancer cell lines and tumors, we analyzed the raw gene expression values of three cell lines and five human tumor microarray expression studies deposited in the NCBI GEO database (Table 1). Except for one case (SW527), all breast cancer cell lines with low SIP1 expression have a high CD24 expression (FIG. 17, Table 2). On the opposite, all cell lines with high SIP1/ZEB2 expression have low CD24 expression. This inverse relationship is confirmed in two breast tumor studies. A similar albeit insignificant trend is observed in a third study, while no significant relation between CD24 and SIP1/ZEB2 expression was detected in the last two studies. As only a small active minority of cancer cells within the tumor might display high SIP1/ZEB2 expression (as in our MDAMB231 cell population), the variability of the tumor expression values could be due to variation of the proportion of SIP1-expressing cells in the samples analyzed. Altogether, these data strongly support our experimental data that only high SIP1 expression is compatible with low CD24 expression in human breast cancer cells.

Moreover, all SIP1/ZEB2-expressing cell lines have enhanced expression of CD44, a cell surface marker used initially to isolate breast cancer stem cells. On the other hand, most, but not all, SIP1/ZEB2-negative cell lines display low CD44 expression. However, since the whole MDAMB231 population stained positively for CD44 by flow cytometry analysis and because no significant correlation between SIP1/ZEB2 expression and CD44 expression was observed in the tumor studies, the value of CD44 as a general cancer stem cell marker seems to be limited. On the other hand, the expression of integrin B1 (ITGB1, CD49f), another cell surface marker used to isolate breast stem cells, is significantly correlated to the expression of SIP1/ZEB2 in all but one study analyzed. A good correlation to SIP1/ZEB2 expression was also observed with a new marker of breast stem cells, ALDH1A1, but only in the tumor studies. A frequent correlation was also observed with PROCR, a marker of skin stem cells, while little or no correlation was seen with CD133, a marker of brain cancer stem cells.

Taken together, our data on CD24, ITGB1, ALDH1A1 and PROCR prove high SIP1 expression contributes to breast cancer sternness.

TABLE 1
study references

| GEO | | | |
|---|---|---|---|
| reference | Author | Reference | Type |
| GSE2109 | Bittner | ExpO International | Tumour |
| | | GenomicConsortium | |
| GSE5764 | Turashvili | BMC Cancer 2007 Mar 27; 7:55. | Tumour |
| | | PMID: 17389037 | |
| GSE3744 | Richardson | Cancer Cell 2006 Feb; 9(2): | Tumour |
| | | 121-32. PMID: 16473279 | |
| GSE9195 | Loi | BMC Genomics 2008 May 22; | Tumour |
| | | 9:239. PMID: 18498629 | |
| GSE6532 | Loi | J Clin Oncol 2007 Apr 1; 25(10): | Tumour |
| | | 1239-46. PMID: 17401012 | |
| GSE12777 | Januario | Genentech | Cell lines |
| GSE10890 | Adai | Genentech | Cell lines |
| GSE10843 | Adai | Genentech | Cell lines |

TABLE 2
Correlation analysis. Pearson coefficient for linear correlation
between expression values for the 203603_s_at (SIP1/ZEB2) and the indicated probes sets are
reported for each study analyzed. Pearson coefficients with a p value below 0.05 are highlighted
in light gray.

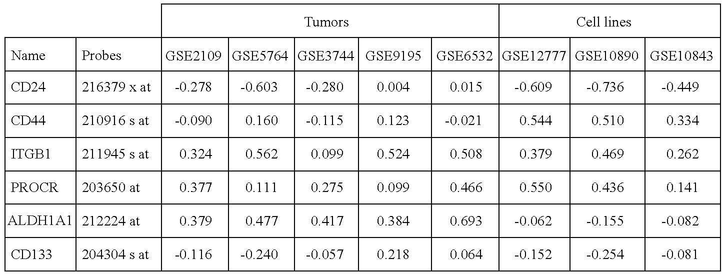

### REFERENCES

Aigner, K., Dampier, B., Descovich, L., Mikula, M., Sultan, A., Schreiber, M., Mikulits, W., Brabletz, T., Strand, D., Obrist, P., Sommergruber, W., Schweifer, N., Wernitznig, A., Beug, H., Foisner, R., and Eger, A. (2007) Oncogene 26(49), 6979-6988.
Bindels, S., Mestdagt, M., Vandewalle, C., Jacobs, N., Volders, L., Noël, A., Van Roy, F., Berx, G., Foidart, J-M. and Gilles, C. (2006). Regulation of vimentin by SIP1 in human epithelial tumor cells. Oncogene 25, 4975-4985.
Brummelkamp, T. R., Bernards, R., and Agami, R. (2002) Cancer Cell 2(3), 243-247.
Cailleau, R., Olive, M., and Cruciger, Q. V. (1978) In vitro 14(11), 911-915.
Comijn, J., Berx, G., Vermassen, P., Verschueren, K, van Grunsven, L., Bruyneel, E., Mareel, M, Huylebroeck, D. and Van Roy, F. (2001). The two handed E box binding zinc finger protein SIP1 down-regulates E-cadherin and induces invasion. Mol. Cell. 7, 1267-1278.
Eger, A., Aigner, K., Sonderegger, S., Dampier, B., Oehler, S., Schreiber, M., Berx, G., Cano, A., Beug, H., and Foisner, R. (2005) Oncogene 24(14), 2375-2385.
Dalerba, P., Cho, R. W. & Clarke, M. F. (2007) Annu. Rev. Med. 58, 267-284.
Geiger, B., Volberg, T., Ginsberg, D., Bitzur, S., Sabanay, I., and Hynes, R. O. (1990) Journal of Cell Science 97 (Pt 4), 607-614
Gupta, G. P., Perk, J., Acharyya, S., de Candia, P., Mittal, V., Todorova-Manova, K., Gerald, W. L., Brogi, E., Benezra, R., and Massague, J. (2007) Proceedings of the National Academy of Sciences of the United States of America.
Henson, D. E. and Patierno, S. R. (2004) Breast Cancer Research and Treatment 87, 291-296.
Hiratsuka, S., Watanabe, A., Aburatani, H. & Maru, Y. (2006) Nat. Cell. Biol. 8, 1369-75.
Itoh, M., Nagafuchi, A., Moroi, S., and Tsukita, S. (1997) The Journal of Cell Biology 138(1), 181-192.
Kaplan, R. N., Riba, R. D., Zacharoulis, S., Bramley, A. H., Vincent, L., Costa, C., MacDonald, D. D., Jin, D. K., Shido, K., Kerns, S. A., Zhu, Z., Hicklin, D., Wu, Y., Port, J. L., Altorki, N., Port, E. R., Ruggero, D., Shmelkov, S. V., Jensen, K. K., Rafii, S. & Lyden, D. (2005) Nature 438, 820-7.
Kang, Y., Siegel, P. M., Shu, W., Drobnjak, M., Kakonen, S. M., Cordon-Cardo, C., Guise, T. A., and Massague, J. (2003) Cancer Cell 3(6), 537-549.
Lacroix, M., and Leclercq, G. (2004) Breast Cancer Research and Treatment 83(3), 249-289.
Muller, S. L., Portwich, M., Schmidt, A., Utepbergenov, D. I., Huber, O., Blasig, I. E., and Krause, G. (2005) The Journal of Biological Chemistry 280(5), 3747-3756.
Schaefer, U., Voloshanenko, O., Willen, D., and Walczak, H. (2007) Front Biosci. 12, 3813-3824.
Sleeman, J. P. (2000) Recent Results Cancer Res. 157, 55-81.
Theard, D., Steiner, M., Kalicharan, D., Hoekstra, D., and van Ijzendoorn, S. C. (2007) Molecular Biology of the Cell 18(6), 2313-2321.
Ui-Tei, K., Naito, Y., Takahashi, F., Haraguchi, T., Ohki-Hamazaki, H., Juni, A., Ueda, R., and Saigo, K. (2004) Nucleic Acids Research 32(3), 936-948.
Vandewalle, C., Comijn, J., De Craene, B., Vermassen, P., Bruyneel, E., Andersen, H., Tulchinsky, E., Van Roy, F. and Berx, G. (2005). SIP1/ZEB2 induces EMT by repressing genes of different epithelial cell-cell junctions. Nucl. Acids Res. 33 (20), 6566-6578.
van Hengel, J., Vanhoenacker, P., Staes, K., and van Roy, F. (1999) Proceedings of the National Academy of Sciences of the United States of America 96(14), 7980-7985.
Wiznerowicz, M., and Trono, D. (2003) Journal of Virology 77(16), 8957-8961.

1. A method of detecting breast cancer stem cells, wherein the method comprises:
utilizing the SIP1 gene and/or the SIP1 gene encoded protein for the detection of breast cancer stem cells.

2. A method of inactivating breast cancer stem cells, the method comprising:
utilizing SIP1 gene repression and/or SIP1 protein inactivation to inactivate breast cancer stem cells.

3. The method according to claim 2, comprising:
inactivating SIP1 protein so as to inactivate the breast cancer stem cells.

4. A method of identifying breast cancer stem cells markers, wherein the method comprises:
utilizing the SIP1 gene and/or the SIP1 gene encoded protein for the identification of breast cancer stem cell markers.

5. The A method of determining breast cancer aggressiveness in a subject, wherein the method comprises:
utilizing the SIP1 gene and/or the SIP1 gene encoded protein for the determination of breast cancer aggressiveness in the subject.

6. A method of lowering breast cancer aggressiveness in a subject suffering therefrom, wherein the method comprises:
utilizing SIP1 gene repression and/or SIP1 protein inactivation to lower breast cancer aggressiveness in the subject.

7. The method of claim 6, comprising:
inactivating SIP1 protein so as to lower breast cancer aggressiveness in the subject.

8. The method of claim 6, comprising repressing SIP1 gene expression so as to lower breast cancer aggressiveness in the subject.

9. The method according to claim 2, comprising repressing SIP1 gene expression so as to inactivate the breast cancer stem cells.
